(19) United States
(12) Patent Application Publication
(10) Pub. No.: US 2004/0192973 A1
(43) Pub. Date: Sep. 30, 2004

(54) Mixed metal oxide catalysts for the production of unsaturated aldehydes from olefins

(75) Inventors: Wugeng Liang, Katy, TX (US); Scott A. Stevenson, Houston, TX (US); James W. Kauffman, Katy, TX (US); John S. Ledford, Richmond, TX (US); Joseph R. Linzer, Houston, TX (US)

Correspondence Address: JIM WHEELINGTON, SABIC Americas, Inc., SABIC Technology Center, 1600 Industrial Blvd., Sugar Land, TX, 77478, US

(73) Assignee: Saudi Basic Industries Corporation

(21) Appl. No.: 10/403,213

(22) Filed: Mar. 31, 2003

(51) Int. Cl. B01J 23/31; C07C 45/28

(52) U.S. Cl. 568/470; 502/311

(57) ABSTRACT

A catalyst for production of unsaturated aldehydes, such as methacrolein, by gas phase catalytic oxidation of olefins, such as isobutylene contains oxides of molybdenum, bismuth, iron, cesium and, optionally, other metals, such as tungsten, cobalt, nickel, antimony, magnesium, zinc and phosphorus. The catalyst has a certain relative amount ratio of cesium to bismuth, a certain relative amount ratio of iron to bismuth and a certain relative amount ratio of bismuth, iron and cesium to molybdenum. For a catalyst of the formula:

Mo₁₂BiₐW_{b}Fe_{c}Co_{d}NiₑSb_{f}Cs_{g}MgₕZnᵢPⱼOₓ

wherein a is 0.1 to 1.5, b is 0 to 4, c is 0.2 to 5.0, d is 0 to 9, e is 0 to 9, f is 0 to 2.0, g is from 0.4 to 1.5, h is 0 to 1.5, i is 0 to 2.0, j is 0 to 0.5 and x is determined by the valences of the other components, c:g=3.3-5.0, c:a=2.0-6.0 and (3a+3c+2d+2e+g+2h+2i)/(2x12+2b)=0.90-1.10.

### BACKGROUND OF THE INVENTION

1. FIELD OF THE INVENTION

This invention relates to a mixed metal oxide catalyst containing oxides of molybdenum, bismuth, iron, cesium and, optionally, other metals for the production of unsaturated aldehydes from olefins, such as methacrolein by gas phase catalytic oxidation of isobutylene in the presence of air or another gas containing molecular oxygen.

2. DESCRIPTION OF THE PRIOR ART

Many catalysts have been disclosed for use in the production of acrolein or methacrolein by catalytic vapor phase oxidation of propylene or isobutylene. U.S. Pat. No. 4,816,603 discloses a catalyst for production of methacrolein and methacrylic acid of the formula:

MoₐW_{b}Bi_{c}Fe_{d}NiₑSb_{f}X_{g}YₕZᵢAⱼOₖ

where X is potassium, rubidium and/or cesium, Y is phosphorus, sulfur, silicon, selenium, germanium and/or boron, Z is zinc and/or lead, A is magnesium, cobalt, manganese and/or tin, a is 12, b is 0.001 to 2, c is 0.01 to 3, d is 0.01 to 8, e is 0.01 to 10, f is 0.01 to 5, g is 0.01 to 2, h is 0 to 5, i is 0.01 to 5, j is 0 to 10 and k is sufficient to satisfy the valences.

U.S. Pat. No. 4,511,671 discloses a catalyst for manufacturing methacrolein of the formula:

MoₐW_{b}Bi_{c}Fe_{d}AₑB_{f}C_{g}DₕOₓ

where A is at least one of nickel and/or cobalt; B is at least one of alkali metals, alkaline earth metals and/or thallium; C is at least one of phosphorus, tellurium, antimony, tin, cerium, lead, niobium, manganese and/or zinc; D is at least one of silicon, aluminum, zirconium, and/or titanium; a is 12, b is 0 to 10, c is 0.1 to 10, d is 0.1 to 20, e is 2 to 20, f is 0 to 10, g is 0 to 4, h is 0 to 30 and x is determined by the atomic valences.

U.S. Pat. No. 4,556,731 discloses a catalyst for production of methacrolein and methacrylic acid of the formula:

AₐB_{b}Fe_{c}S_{c}MₑMo₁₂Oₓ

where A is an alkali metal, such as potassium, rubidium, cesium or mixtures thereof, thallium, silver or mixtures thereof, B is cobalt, nickel, zinc, cadmium, beryllium, calcium, strontium, barium, radium or mixtures thereof, X is bismuth, tellurium or mixtures thereof and M is (1) Cr+W, Ge+W, Mn+Sb, Cr+P, Ge+P, Cu+W, Cu+Sn, Mn+Cr, Pr+W, Ce+W, Sn+Mn, Mn+Ge or combinations thereof, (2) Cr, Sb, Ce, Pn, Ge, B, Sn, Cu or combinations thereof, or (3) Mg+P, Mg+Cu, Mg+Cr, Mg+Cr+W, Mg+W, Mg+Sn or combinations thereof, a is 0 to 5, b is 0 to 20, c is 0 to 20, d is 0 to 20, e is 0.01 to 12 and x satisfies the valence requirements.

U.S. Pat. No. 5,245,083 discloses a catalyst for preparing methacrolein of a mixture of composition (1) of the formula:

MoₐB_{b}Fe_{c}XₑZ_{f}O_{g}

where X is Ni and/or Co, Z is at least one of W, Be, Mg, S, Ca, Sr, Ba, Te, Se, Ce, Ge, Mn, Zn, Cr, Ag, Sb, Pb, As, B, P, Nb, Cu, Cd, Sn, Al, Zr and Ti, a is 12 b is 0.1 to 10, c is 0 to 20, d is 0 to 20, f is 0 to 4 and g satisfies the valence requirement and composition (2) of the formula:

AₘMoₐOₚ

where A is at least one of K, Rb and Cs, m is 2, n is 1 to 9 and p is 3n+1.

U.S. Pat. No. 5,138,100 discloses a catalyst for preparing methacrolein with composition (1) of the formula:

MoₐBi_{b}Fe_{c}X_{d}YₑZ_{f}O_{g}

where X is at least one of Ni and Co, Y is at least one of K, Rb, Cs and Tl, Z is at least one of the elements belonging to Groups 2, 3, 4, 5, 6, 7, 11, 12, 13, 14, 15 and 16, specifically beryllium, magnesium, calcium, strontium, barium, titanium, zirconium, cerium, niobium, chromium, tungsten, manganese, copper, silver, zinc, cadmium, boron, aluminum, germanium, tin, lead, phosphorus, arsenic, antimony, sulfur, selenium and tellurium, a is 12, b is 0.1 to 10, c is 0 to 20, d is 0 to 20, e is 0 to 2, f is 0 to 4, and g satisfies the valence requirement and composition (2) of the formula:

LnₕMoₐOⱼ

where Ln is at least one of the rare earth elements, h is 0.2 to 1.5, i is 1 and j satisfies the valence requirement. The atomic ratio of the rate earth element to molybdenum is disclosed to be in the range from 0.2 to 1.5 with an atomic ratio less than 0.2 resulting in high selectivity but poor activity and with an atomic ratio greater than 1.5 resulting in high activity but poor selectivity.

U.S. Pat. No. 4,537,874 discloses a catalyst for production of unsaturated aldehydes of the formula:

BiₐW_{b}Fe_{c}Mo_{d}AₑB_{f}C_{g}DₕOₓ

where A is nickel and/or cobalt, B is at least one of alkali metal, alkaline earth metals and thallium, C is at least one of phosphorus, arsenic, boron, antimony, tin, cerium, lead and niobium, D is at least one of silicon, aluminum, zirconium and titanium, a is 0.1 to 10.0, b is 0.5 to 10.0, c is 0.1 to 10.0, d is 12, e is 2.0 to 20.0, f is 0.001 to 10.0, g is 0 to 10.0 and h satisfies the valence requirement. The ratio of a/b is 0.01 to 6.0 so that bismuth is combined very stably with tungsten and compounds such as bismuth trioxide and bismuth molybdate are not formed.

U.S. Pat. No. 5,728,894 discloses a catalyst for producing methacrolein of the formula:

Mo₁₂BiₐCe_{b}K_{c}Fe_{d}AₑB_{f}Oₓ

where A is Co or a mixture of Co and Mg having an atomic ratio of not more than 0.7, B is Rb, Cs or a mixture thereof, a is 0 to 8, b is 0 to 8, c is 0 to 1.2, c is 0 to 2.5, e is 1.0 to 12, f is 0 to 2.0, g satisfies the valence requirement. The relative atomic ratio of iron to bismuth and cerium should be 0<d/(a+b+d)≦0.9. The relative atomic ratio of bismuth, cerium and potassium should be 0.05≦b/(a+b+c)≦0.7. The relative atomic ratio of potassium to bismuth and cerium should be 0<c/(a+b+c)≦0.4. Bismuth, cerium, potassium, iron and cobalt are indispensable elements for the disclosed invention.

Prior art discloses mixed metal oxide catalysts which contain molybdenum, bismuth, iron, cesium and other metals for the production of methacrolein. Furthermore, prior art discloses certain ranges of amounts of these metals. Some prior art discloses relative ratios of certain components to other components. The effect of the selection of certain components for a mixed metal oxide catalyst for the production of methacrolein and the relative relationship of some of these components to other components has not been investigated in complete detail.

### SUMMARY OF THE INVENTION

The present invention is for a catalyst of the general formula:

Mo_{z}BiₐFe_{c}Cs_{g}Oₓ

a is in the range from 0.1 to 1.5, c is in the range from 0.2 to 5.0, g is in the range from 0.1 to 1.5 and x is determined by the valences of the other components. The catalyst of the present invention has a relative amount ratio of iron to cesium which is in the range of 3.3 to 5.0, i.e., c:g=3.3-5.0, a relative amount ratio of iron to bismuth which is in the range of 2.0 to 6.0, i.e., c:a=2.0-6.0 and a relative amount of bismuth, iron and cesium to molybdenum represented by the formula (3a+3c+g)/(2×z) =0.90-1.10 from which the range for z can be determined by selecting values for a, c and g.

The process of making the catalyst is generally to dissolve the metal compounds of molybdenum, bismuth, iron, cesium and, optionally, other metals, such as tungsten, cobalt, nickel, antimony, magnesium, zinc, phosphorus, potassium, rubidium, thallium, manganese, barium, chromium, boron, sulfur, silicon, aluminum, titanium, cerium, tellurium, tin, vanadium, zirconium, lead, cadmium, copper and niobium, and precipitate a catalyst precursor which is calcined to form a mixed metal oxide catalyst. The metal compounds may be salts (e.g., nitrates, halides, ammonium, organic acid, inorganic acid), oxides, hydroxides, carbonates, oxyhalides, sulfates and other groups which may exchange with oxygen under high temperatures so that the metal compounds become metal oxides. Preferably, the metal compounds are soluble in water or an acid. It is preferable that the molybdenum compound and the tungsten compound are ammonium salts, that the phosphorus compound is phosphoric acid, and that the bismuth compound, the ferric compound, the nickel compound, the cobalt compound, the magnesium compound, the zinc compound, the cesium compound, the potassium compound, the rubidium compound, the thallium compound, the manganese compound, the barium compound, the chromium compound, the boron compound, the sulfur compound, the silicon compound, the aluminum compound, the titanium compound, the cerium compound, the tellurium compound, the tin compound, the vanadium compound, the zirconium compound, the lead compound, the cadmium compound, the copper compound and the niobium compound are nitrates, oxides or acids and the antimony compound is an oxide.

The process of using the catalyst is generally to contact propylene or isobutylene and a molecular oxygen-containing gas in the presence of the catalyst of the present invention. This process is a gas phase catalytic oxidation of an olefin to an aldehyde. The use of the catalyst of the present invention in this process increases activity and selectivity to the production of methacrolein.

### DESCRIPTION OF EMBODIMENTS OF THE INVENTION

According to the present invention, a catalyst is provided for producing acrolein or methacrolein by oxidation of propylene or isobutylene. The oxidation is a catalytic reaction that converts an olefin in the presence of oxygen to an unsaturated aldehyde and water:

H₂C═CA—CH₃+O₂→H₂C═CA—CHO+H₂O

where A is hydrogen or an alkyl group. Carboxylic acid is also produced in a side reaction.

The catalyst is a mixed metal oxide of the formula:

Mo_{z}BiₐFe_{c}Cs_{g}Oₓ

wherein a is in the range from 0.1 to 1.5, c is in the range from 0.2 to 5.0, g is in the range from 0.1 to 1.5 and x is determined by the valences of the other components. For a catalyst for producing methacrolein by oxidation of isobutylene, it is preferred that g is in the range from 0.4 to 1.5

In addition, a mixed metal oxide catalyst of the present invention has a relative amount ratio of iron to cesium which is in the range of 3.3 to 5.0, i.e., c:g=3.3-5.0, a relative amount ratio of iron to bismuth which is in the range of 2.0 to 6.0, i.e., c:a=2.0-6.0 and a relative amount ratio of bismuth, iron and cesium to molybdenum which is represented by the formula (3a+3c+g)/(2×z)=0.90-1.10 from which the range for z can be determined by selecting values for a, c and g.

U.S. Pat. No. 5,728,894 discloses three relationships of relative atomic ratios:

iron to bismuth and cerium: 0<d/(a+b+d)≦0.9

bismuth, cerium and potassium: 0.05≦b/(a+b+c)≦0.7

potassium to bismuth and cerium: 0<c/(a+b+c)≦0.4

The first relationship of relative atomic ratios defines the relative amount of iron to bismuth. Based on this relationship, the iron content should be less than the bismuth content, which can be seen in the examples of U.S. Pat. No. 5,728,894. The relationship disclosed in the present invention is to the contrary in that the iron content is greater than the bismuth content, i.e., c:a=2.0-6.0.

The second relationship of relative atomic ratios defines the relative amount of cerium content. Cerium is not a required element of the catalyst of the present invention. U.S. Pat. No. 5,728,894 discloses cerium as an indispensable element of this prior art catalyst.

The third relationship of relative atomic ratios defines the relative amount of alkaline metals in the catalyst to the bismuth content. In the catalyst of the present invention, the relative amount of alkaline metals to bismuth is greater than the upper limit disclosed in U.S. Pat. No. 5,728,894 (0.4).

In the catalyst of U.S. Pat. No. 5,728,894, cerium, potassium and cobalt are indispensable elements while in the catalyst of the present invention these elements are optional.

Preferably, the catalyst of the present invention is of the formula:

Mo₁₂BiₐW_{b}Fe_{c}Cs_{g}MₘO^{x}

wherein M is one or more selected from cobalt, nickel, magnesium, zinc, potassium, rubidium, thallium, manganese, barium, chromium, cerium, tin, lead, cadmium and copper, m is in the range from 0 to 9 and b is 0 to 9. If any of these metals are components of the catalyst, the relative amount ratio of bismuth, iron, cesium, and any and all M metals to molybdenum and tungsten represented by the formula (3a+3c+g+Σvₙmₙ)/(2x12+2b)=0.90-1.10 wherein ΣVₙmₙ is the sum of the product of the valence (v) and the relative amount of each M (m), n being an integer corresponding to each M metal present in the catalyst. For example, if M were nickel (v=2, n=1) and cobalt (v=2, n=2) present in the amounts of 4.0 and 0.5, respectively, Σvₙmₙ would be (2×4.0)+(2×0.5)=9.

More preferably, the catalyst is of the formula:

Mo₁₂BiₐW_{b}Fe_{c}Cs_{g}MₘM′_{m′}O_{X}

wherein M′ is one or more of antimony, phosphorus, boron, sulfur, silicon, aluminum, titanium, tellurium, vanadium, zirconium and niobium and m′ is from 0 to 9. M′ and m′ would not be taken into account in the formulae above for relative amounts of components.

Most preferably, the catalyst is of the formula:

Mo₁₂BiₐW_{b}Fe_{c}Co_{d}NiₑSb_{f}Cs_{g}MgₕZnᵢPⱼOₓ

wherein b is 0 to 4, d is 0 to 9, e is 0 to 9, f is 0 to 2.0, h is 0 to 1.5, i is 0 to 2.0, j is 0 to 0.5. Preferably, c:g is in the range of 3.3-4.8, c:a is in the range of 2.4-4.8, and the relative amount of bismuth, iron, cesium, cobalt, nickel, magnesium, and/or zinc to molybdenum and tungsten is represented by the formula(3a+3c+2d+2e+g+2h+2i)/(2x12+2b)=0.91-1.09.

The process of making the catalyst is generally to dissolve the metal compounds dissolved in water or in an acid and precipitate a catalyst precursor which is calcined to form a mixed metal oxide catalyst. The metal compounds may be salts (e.g., nitrates, halides, ammonium, organic acid, inorganic acid), oxides, hydroxides, carbonates, oxyhalides, sulfates and other groups which may exchange with oxygen under high temperatures so that the metal compounds become metal oxides. Preferably, the metal compounds are soluble in water or an acid. It is more preferred that the molybdenum compound and the tungsten compound are ammonium salts, such as ammonium paramolybdate or ammonium molybdate and ammonium paratungstate or ammonium tungstate, respectively, that the phosphorus compound is phosphoric acid, that the bismuth, iron, cobalt, nickel, cesium, magnesium, zinc, phosphorus, potassium, rubidium, thallium, manganese, barium, chromium, boron, sulfur, silicon, aluminum, titanium, cerium, tellurium, tin, vanadium, zirconium, lead, cadmium, copper and niobium compounds are nitrates, oxides or acids and that the antimony compound is an oxide, such as antimony oxide or antimony trioxide. For bismuth, iron, cesium, cobalt, nickel, magnesium and zinc compounds, it is preferred that they are nitrates.

The present invention does not depend on a particular order of addition of the components. While a particular order of addition of the various metal compound components may affect the performance of the catalyst, the present invention is directed toward the particular relative amount of certain components to other components without regard to the order in which the steps in the process of making the catalyst occur.

An example of making the catalyst of the claimed invention is to dissolve an ammonium salt of molybdenum, such as ammonium paramolybdate or ammonium molybdate and, optionally, an ammonium salt of tungsten, such as ammonium paratungstate or ammonium tungstate, and phosphoric acid in water, dissolve a bismuth nitrate in an acid, dissolve a iron nitrate and, optionally, a cobalt nitrate, a nickel nitrate, a magnesium nitrate, and a zinc nitrate in water or in the acid with the bismuth nitrate, mix the solutions at a temperature in the range from 40° C. to 100° C., preferably 60° C. to 95° C., to obtain a precipitate to form a slurry and then add a cesium nitrate and, optionally, an antimony oxide to the slurry while maintaining the temperature. The cesium nitrate and the antimony oxide may be added to the slurry as solids. The slurry may be aged for 2 to 24 hours, preferably 8 to 18 hours, most preferably 5 to 10 hours. The liquid of the slurry is removed by evaporation and the solid precipitate is dried and calcined to obtain a catalyst. The liquid may be removed and the solid precipitate dried at the same time by spray drying. The liquid may be evaporated at a temperature of 500 to 125° C.

Drying of the catalyst precursor may be in air or an inert gas and in an oven or a spray dryer. Preferably, drying is in an oven in air at a temperature of 100-150° C. for 2-5 hours

One purpose of calcination of the catalyst precursor is to obtain an oxide of the metal components. The catalyst precursor may be calcined at a temperature of 200-600° C. for 1-12 hours. Calcination may be in two stages, one at a temperature of 150-400° C. for 1-5 hours and another at a temperature of 460-600° C. for 4-8 hours. For a two-stage calcination, preferably, the first is at a temperature of 290-310° C. for 2 hours and second at a temperature of 460-500° C. for 6 hours. Denitrification may occur in the first step. In the alternative, calcination is in one stage at a temperature of 485° C. for 2 hours with a temperature ramp of up to 10° C./min from ambient temperature to 485° C. instead of an initial step or denitrification at a temperature of 300° C. for two hours. Calcination may be done in a high temperature oven or kiln.

The catalyst may be processed by sieving, forming and other means known in the art to obtain catalyst particles of a certain size. Desired particle size and particle size distribution are related to the design of the reactor (size, shape, configuration, etc.), to the pressure drop intended for the process and to the process flow. For a two stage calcination, the catalyst may be sieved or formed after the first stage calcination and before the second stage calcination. In a commercial process the catalyst precursor may be sieved and formed after spray drying and before calcination.

The X-ray diffraction pattern of the mixed metal oxide compounds is descriptive of the catalyst made by the process of the present invention. The catalyst compositions of the Examples above have a characteristic X-ray diffraction having diffraction peaks at the diffraction angles of 2θ, measured by using Cu Kα radiation, at 25.5, 26.6 and 28.0 (+/−0.1°). There may be several additional diffraction peaks present in a catalyst composition of the present invention but these peaks will normally be evident.

The catalyst of the present invention may be used as an unsupported catalyst or a supported catalyst. The surface area of an unsupported catalyst is from 0.1 to 150m²/g, preferably from 1 to 20 m²/g. If supported, the support should be an inert solid which is chemically unreactive with any of the active components of the catalyst and is preferably silica, alumina, niobia, titania, zirconia or mixtures thereof. The catalyst may be affixed to the support by methods known in the art, including incipient wetness, slurried reactions and spray drying. The catalyst is not limited by shape, size or particle distribution and may be formed as appropriate for the reaction vessel in the process. Examples are powder, granules, spheres, cylinders, saddles, etc.

The catalyst is used in the gas phase catalytic oxidation of a feedstock gas comprising propylene or isobutylene, oxygen, water and an inert gas, such as nitrogen, to produce acrolein or methacrolein. Oxygen may be supplied in the pure form or in an oxygen containing gas, such as air or as an oxygen-diluent gas mixture. The diluent gas may be nitrogen, a hydrocarbon which is gaseous under the process conditions or carbon dioxide. The reaction temperature is preferably from 250-450° C., most preferably 370-410° C. The reactor may be a fixed bed or a fluidized bed reactor. Reaction pressure may be from 0 to 100 psig. Space velocity may be from 800 to 8000 hr⁻¹.

The invention having been generally described, the following examples are given as particular embodiments of the invention and to demonstrate the practice and advantages thereof. It is understood that the examples are given by way of illustration and are not intended to limit the specification or the claims to follow in any manner.

### EXAMPLE 1

43.57 g of ammonium paramolybdate and 1.65 g of ammonium paratungstate were added into 87 ml of de-ionized water. The mixture was stirred and heated to 95° C. to form a solution.

A second solution was prepared by adding 1.3 ml of 70% nitric acid to 9.3 ml of de-ionized water. 9.98 g of bismuth nitrate was dissolved in the nitric acid solution. To this solution was added 19.94 g of ferric nitrate, 23.92 g nickel nitrate, 12.03 g of cobalt nitrate, 2.64 g of magnesium nitrate, 3.24 g of zinc nitrate and 85.3 ml of de-ionized water.

The second solution was added to the first solution dropwise. Precipitates were formed during the addition which created a slurry. 2.41 g of cesium nitrate and 2.11 g of antimony oxide were added as solids to the slurry.

The slurry was aged for 10 hours at 80° C. while being stirred. After aging, the liquid was evaporated at 100° C. The solid was dried at 120° C. for 3 hours. The dried solid was calcined at 300° C. for 2 hours in flowing air. The calcined solid was sieved to a mesh size of 20-30. The sieved solid was calcined at 500° C. for 6 hours in flowing air. A catalyst of the following composition was obtained: Mo₁₂Bi_{1.0}W_{0.3}Fe_{2.4}Co_{2.0}Ni_{4.0}Sb_{0.7}Cs_{0.6}Mg_{0.5}Zn_{0.5}.

### EXAMPLE 2

The procedure of Example 1 was repeated except the amount of bismuth nitrate was 5.20 g, so that the composition of the catalyst was Mo₁₂Bi_{0.5}W_{0.3}Fe_{2.4}Co_{2.0}Ni_{4.0}Sb_{0.7}CS_{0.6}Mg_{0.5}Zn_{0.5}.

### EXAMPLE 3

The procedure of Example 1 was repeated except the amount of the bismuth nitrate was 11.6 g and the amount of the ferric nitrate was 23.4 g so that the composition of the catalyst was Mo₁₂Bi_{1.2}W_{0.3}Fe_{2.9}Co_{2.0}Ni_{4.0}Sb_{0.7}CS_{0.6}Mg_{0.5}Zn_{0.5}.

### EXAMPLE 4

The procedure of Example 1 was repeated except the amount of ammonium paramolybdate was 45.01 g, the amount of the amount of nickel nitrate was 36.0 g, the amount of magnesium nitrate was 5.21 g, cobalt nitrate, zinc nitrate and ammonium paratungstate were not present and the ammonium molybdate solution was heated to 95° C. over 45 minutes before the second solution was added and the catalyst precursor 485° C. with a 10° C./min ramp so that the composition of the catalyst was Mo₁₂Bi1.2Fe_{2.4}Ni_{6.0}Sb_{0.7}Cs_{0.6}Mg_{1.0}.

### EXAMPLE 5

The procedure of Example 1 was repeated except the amount of bismuth nitrate was 5.98 g and the amount of ferric nitrate was 11.57 g so that the composition of the catalyst was Mo₁₂Bi_{0.6}W0.3Fe_{2.0}Co_{2.0}Ni_{4.0}Sb_{0.7}Cs_{0.6}Mg_{0.5}Zn_{0.5}.

### EXAMPLE 6

The procedure of Example 1 was repeated except the amount of bismuth nitrate was 6.25 g and the amount of ferric nitrate was 17.4 g so that the composition of the catalyst was Mo₁₂Bi_{0.6}W_{0.3}Fe_{2.0}Co_{2.0}Ni_{4.0}Sb_{0.7}Cs_{0.6}Mg_{0.5}Zn_{0.5}.

### EXAMPLE 7

The procedure of Example 1 was repeated except no ammonium paratungstate was added so that the composition of the catalyst was Mo₁₂Bi_{1.0}Fe_{2.4}Co_{2.0}Ni_{4.0}Sb_{0.7}Cs_{0.6}Mg_{0.5}Zn_{0.5}.

### EXAMPLE 8

The procedure of Example 1 was repeated except no ammonium paratungstate was added and 44.7 g of ammonium molybdate was added so that the composition of the catalyst was Mo_{12.3}Bi_{1.0}Fe_{2.4}Co_{2.0}Ni_{4.0}Sb_{0.7}Cs_{0.6}Mg_{0.5}Zn_{0.5}.

### COMPARATIVE EXAMPLE 1

The procedure of Example 1 was repeated except the amount of bismuth nitrate was 2.65 g so that the composition of the catalyst was Mo₁₂Bi_{0.25}W_{0.3}Fe_{2.4}Co_{2.0}Ni_{4.0}Sb_{0.7}CS_{0.6}Mg_{0.5}Zn_{0.5}.

### COMPARATIVE EXAMPLE 2

The procedure of Example 1 was repeated except the amount of bismuth nitrate was 14.39 g so that the composition of the catalyst was Mo₁₂Bi_{1.5}W_{0.3}Fe_{2.4}Co_{2.0}Ni_{4.0}Sb_{0.7}Cs_{0.6}Mg_{0.5}Zn_{0.5}.

### COMPARATIVE EXAMPLE 3

The procedure of Example 1 was repeated except the amount of cesium nitrate was 4.68 g so that the composition of the catalyst was Mo₁₂Bi_{1.0}W_{0.3}Fe_{2.4}Co_{2.0}Ni_{4.0}Sb_{0.7}Cs_{1.2}Mg_{0.5}Zn_{0.5}.

### COMPARATIVE EXAMPLE 4

The procedure of Example 1 was repeated except the amount of bismuth nitrate was 8.12 g, the amount of ferric nitrate was 16.91 g and the amount of cesium nitrate was 16.91 g so that the composition of the catalyst was Mo₁₂Bi_{0.8}W_{0.3}Fe_{2.0}Co_{2.0}Ni_{4.0}Sb_{0.7}Cs_{0.8}Mg_{0.5}Zn_{0.5}.

### COMPARATIVE EXAMPLE 5

The procedure of Example 1 was repeated except the amount of bismuth nitrate was 11.24 g, the amount of ferric nitrate was 22.63 g and the amount of cesium nitrate was 1.89 g so that the composition of the catalyst was Mo₁₂Bi_{1.2}W_{0.3}Fe_{2.9}Co_{2.0}Ni_{4.0}Sb_{1.5}Cs_{0.5}Mg_{0.5}Zn_{0.5}.

### COMPARATIVE EXAMPLE 6

The procedure of Example 1 was repeated except the amount of ferric nitrate was 10.31 g so that the composition of the catalyst was Mo₁₂Bi_{1.0}W_{0.3}Fe_{1.2}Co_{2.0}Ni_{4.0}Sb_{0.7}Cs_{0.6}Mg_{0.5}Zn_{0.5}.

### COMPARATIVE EXAMPLE 7

The procedure of Example 1 was repeated except the amount of ammonium paramolybdate was 54.46 g so that the composition of the catalyst was Mo₁₅Bi_{1.0}W_{0.3}Fe_{1.2}Co_{2.0}Ni_{4.0}Sb_{0.7}Cs_{0.6}Mg_{0.5}Zn_{0.5}.

### COMPARATIVE EXAMPLE 8

The procedure of Example 1 was repeated except the amount of ammonium paramolybdate was 32.86 g so that the composition of the catalyst was Mo₉Bi_{1.0}W_{0.3}Fe_{2.4}Co_{2.0}Ni_{4.0}Sb_{0.7}Cs_{0.6}Mg_{0.5}Zn_{0.5}.

For each of the catalysts from the Examples above, 1.0-2.0 cc of catalyst were mixed with quartz chips to make a total volume of 5 cc, which were placed into a downflow reactor having an internal diameter of 0.25 inches. A gas consisting of 3.9% isobutylene, 8.4% oxygen, 28% water and the balance as nitrogen was passed over the catalyst bed in the reactor. The volumetric flow rates were varied between 38 and 85 sccm. The internal reactor temperature was maintained at 390° C. The catalyst loading and gas flow rate were adjusted such that, where possible, a conversion between 97 and 99% was obtained. Product liquid was condensed into a glass trap maintained at 0° C. for a period of approximately three hours. The yields of methacrylic acid and acetic acid were determined from this liquid. The concentrations of isobutylene, methacrolein and other byproducts were determined from on-line analysis by gas chromatography.

Catalyst activities are reported in Table I relative to example 6, for which 1.5 cc of catalyst at a flow rate of 38 sccm gave 97.7% conversion, 89.1% selectivity to methacrolein and methacrylic acid, and a one-pass yield of 87.0%. Repeated tests of example 6 suggest that the accuracy of the activity measurement is roughly ±5%.

It is well known that selectivity for isobutylene oxidation (and indeed most partial oxidation reactions) is dependent on isobutylene conversion; as conversion is increased the selectivity decreases due to further oxidation of the desired products. Given this, the selectivities of two different catalysts must be compared at the same conversion for the comparison to be meaningful. We measured the selectivity of example 1 across a wide range of conversions, from less than 30% to more than 99% and fit a curve to this data over that range. The actual selectivities of examples 2 through 8 and comparative examples 1 through 8 were compared to the selectivity curve that was generated for the catalyst of example 1 at the same conversion. The absolute per cent difference between the selectivities of the catalysts of examples 2 through 8 and comparative examples 1 through 8 and the selectivity of example 1 at the same conversion is reported in Table 1 as “relative selectivity.” The measurement error on the selectivity comparison is roughly ±1%. Mass balances were measured for every sample and averaged 96%.

TABLE I
Mo₁₂BiₐW_{b}Fe_{c}Co_{d}NiₑSb_{f}Cs_{g}MgₕZnᵢPⱼOₓ

| | | | (3a + 3c + | RELA- | |
|---|---|---|---|---|---|
| | | | 2d + 2e + | TIVE | |
| | | | g + 2h + 2i)/ | ACTIV- | RELATIVE |
| EXAMPLE | c/a | c/g | (2 × 12 + 2b) | ITY | SELCTIVITY |
| 1 | 2.4 | 4.0 | 1.01 | 1.24 | same |
| 2 | 4.8 | 4.0 | 0.95 | 1.64 | same |
| 3 | 2.4 | 4.8 | 1.09 | 1.14 | same |
| 4 | 2.4 | 4.0 | 1.06 | 1.15 | same |
| 5 | 3.3 | 3.3 | 0.91 | 1.10 | +1 |
| 6 | 3.3 | 3.3 | 0.91 | 1.00 | same |
| 7 | 2.4 | 4.0 | 1.03 | 1.02 | same |
| 8 | 2.4 | 4.0 | 1.0 | 1.53 | +1 |
| COMPARATIVE 1 | 9.6 | 4.0 | 0.92 | 2.25 | −1 |
| COMPARATIVE 2 | 1.7 | 4.0 | 1.07 | 0.65 | same |
| COMPARATIVE 3 | 2.4 | 2.0 | 1.03 | 0.45 | same |
| COMPARATIVE 4 | 2.5 | 2.5 | 0.94 | 0.96 | −2 |
| COMPARATIVE 5 | 2.4 | 5.8 | 1.09 | 1.48 | −4 |
| COMPARATIVE 6 | 1.2 | 2.0 | 0.86 | 0.91 | −2 |
| COMPARATIVE 7 | 2.4 | 4.0 | 0.81 | 1.62 | −2 |
| COMPARATIVE 8 | 2.4 | 4.0 | 1.33 | 0.14 | −3 |

The above examples demonstrate the effectiveness of the relative amount ratios of certain components to certain other components in a mixed metal oxide catalyst for the catalytic oxidation of an olefin to an unsaturated aldehyde, e.g., propylene or isobutylene to acrolein or methacrolein. Those catalysts which have ratios of iron to bismuth, iron to cesium and bismuth, iron, cobalt, nickel, cesium, magnesium and zinc to molybdenum and tungsten which are within the ranges of 2.4-4.8, 3.3-4.8, and 0.91-1.09, respectively, have selectivity as good or better than those catalysts having any one of these ratios outside the ranges of 2-6, 3.3-5 and 0.9 to 1.1, respectively. The catalyst which has a ratio outside the ranges of 2-6, 3.3-5 and 0.9 to 1.1, respectively, and has selectivity as good as that of catalysts of the present invention (Comparative Example 2) has activity which is unsuitable for good catalyst performance (0.65 relative activity). Those catalysts which have ratios outside the ranges of 2-6, 3.3-5 and 0.9 to 1.1, respectively, and have activity higher than that of catalysts of the present invention (Comparative Examples 1, 5 and 7 at 2.25, 1.48 and 1.62 relative activity) have selectivities which are unsuitable for good catalyst performance (−1, −4 and −2 relative selectivity).

Obviously, numerous modifications and variations of the present invention are possible in light of the above teachings. It is therefore to be understood that within the scope of the appended claims, the invention may be practiced otherwise than as specifically described herein.

What is claimed is:

1. A catalyst for the oxidation of an olefin to an unsaturated aldehyde comprising a mixed metal oxide of the formula:
Mo_{z}BiₐFe_{c}Cs_{g}Oₓ
wherein a is in the range from 0.1 to 1.5, c is in the range from 0.2 to 5.0, g is in the range from 0.1 to 1.5, x is determined by the valences of the other components and wherein the relative amount ratio of c to g is from 3.3 to 5.0, the relative amount ratio of c to a is from 2.0 to 6.0 and the relative amount ratio of (3a+3c+g)/(2×z) is from 0.90 to 1.10.

2. The catalyst of claim 1 wherein g is in the range from 0.4 to 1.5.

3. The catalyst of claim 1 wherein the mixed metal oxide is of the formula:
Mo₁₂BiₐW_{b}Fe_{c}Cs_{g}MₘOₓ
wherein M is one or more selected from cobalt, nickel, magnesium, zinc, potassium, rubidium, thallium, manganese, barium, chromium, cerium, tin, lead, cadmium and copper, m is in the range from 0 to 9 and b is in the range from 0 to 9 and
wherein the relative amount ratio of (3a+3c+g+Σvₙmₙ)/(2x12+2b) is from 0.90 to 1.10 with v being valence of each M and n being an integer for each M.

4. The catalyst of claim 3 wherein the mixed metal oxide is of the formula:
Mo₁₂BiₐW_{b}Fe_{c}Cs_{g}MₘM′_{m′}Oₓ
wherein M′ is one or more selected from antimony, phosphorus, boron, sulfur, silicon, aluminum, titanium, tellurium, vanadium, zirconium and niobium, m′ is in the range from 0 to 9.

5. The catalyst of claim 4 wherein the mixed metal oxide is of the formula:
Mo₁₂BiₐW_{b}Fe_{c}Co_{d}NiₑSb_{f}Cs_{g}MgₕZnᵢPⱼOₓ
wherein b is 0 to 4, d is 0 to 9, e is 0 to 9, f is 0 to 2.0, h is 0 to 1.5, i is 0 to 2.0, j is 0 to 0.5 and
wherein the relative amount ratio of (3a+3c+2d+2e+g+2h+2i)/(2x12+2b) is from 0.90 to 1.10.

6. The catalyst of claim 5 wherein the relative amount ratio of c to a is from 2.4 to 4.8, the relative amount ratio of c to g is from 3.3 to 4.8 and the relative amount ratio of (3a+3c+2d+2e+g+2h+2i)/(2x12+2b) is from 0.91 to 1.09.

7. The catalyst of claim 5 wherein the catalyst has an X-ray diffraction pattern of diffraction peaks at the diffraction angles of 2θ, measured by using Cu Kα radiation, at 25.5, 26.6 and 28.0.

8. The catalyst of claim 5 wherein the catalyst is unsupported and has a surface area of from 0.1 to 150m²/g.

9. The catalyst of claim 8 wherein the catalyst has a surface area of from 1 to 20 m²/g.

10. The catalyst of claim 1 wherein the mixed metal oxide is supported on an inert support.

11. The catalyst of claim 10 wherein the inert support is silica, alumina, niobia, titania, zirconia or mixtures thereof.

12. The catalyst of claim 10 wherein the catalyst is formed into powder, granules, spheres, cylinders or saddles.

13. A process for preparing catalyst for the oxidation of an olefin to an unsaturated aldehyde comprising:
a) dissolving molybdenum, bismuth, iron and cesium metal compounds in water or acid;
b) precipitating a catalyst precursor;
c) removing liquid form a solid;
d) drying the solid; and
e) calcining the solid to form oxides of the metals to form a catalyst of the general formula:
Mo_{z}BiₐFe_{c}Cs_{g}Oₓ
wherein a is in the range from 0.1 to 1.5, c is in the range from 0.2 to 5.0, g is in the range from 0.1 to 1.5, x is determined by the valences of the other components and wherein the relative amount ratio of c to g is from 3.3 to 5.0, the relative amount ratio of c to a is from 2.0 to 6.0 and the relative amount ratio of (3a+3c+g)/(2×z) is from 0.90 to 1.10.

14. The process of claim 13 wherein the molybdenum compound is an ammonium salt.

15. The process of claim 14 wherein the molybdenum compound is ammonium paramolybdate or ammonium molybdate.

16. The process of claim 13 further comprising a tungsten compound.

17. The process of claim 16 wherein the tungsten compound is an ammonium salt.

18. The process of claim 17 wherein the tungsten compound is ammonium paratungstate or ammonium tungstate.

19. The process of claim 13 wherein the bismuth compound is a nitrate.

20. The process of claim 13 wherein the iron compound is a nitrate.

21. The process of claim 13 further comprising a cobalt compound.

22. The process of claim 21 wherein the cobalt compound is a nitrate.

23. The process of claim 13 further comprising a nickel compound.

24. The process of claim 23 wherein the nickel compound is a nitrate.

25. The process of claim 13 further comprising an antimony compound.

26. The process of claim 25 wherein the antimony compound is an oxide.

27. The process of claim 13 wherein the cesium compound is a nitrate.

28. The process of claim 13 further comprising a zinc compound.

29. The process of claim 28 wherein the zinc compound is a nitrate.

30. The process of claim 13 comprising a compound of M wherein M is one or more selected from cobalt, nickel, magnesium, zinc, potassium, rubidium, thallium, manganese, barium, chromium, cerium, tin, lead, cadmium and copper and wherein the catalyst is of the formula:
Mo₁₂BiₐW_{b}Fe_{c}Cs_{g}MₘOₓ
wherein m is 0 to 9 and b is 0 to 9 and
wherein the relative amount ratio of (3a+3c+g+Σvₙmₙ) /(2x12+2b) is from 0.90 to 1.10 with v being valence of each M and n being an integer for each M.

31. The process of claim 30 comprising a compound of M′ wherein M′ is one or more selected from antimony, phosphorus, boron, sulfur, silicon, aluminum, titanium, tellurium, vanadium, zirconium and niobium, and wherein the catalyst is of the formula:
MO₁₂BiₐW_{b}Fe_{c}Cs_{g}MₘM′_{m′}Oₓ
m′ is in the range from 0 to 9.

32. The process of claim 31 wherein catalyst is of the formula:
Mo₁₂BiₐW_{b}Fe_{c}Co_{d}NiₑSb_{f}Cs_{g}MgₕZnᵢPⱼOₓ
wherein b is 0 to 4, d is 0 to 9, e is 0 to 9, f is 0 to 2.0, h is 0 to 1.5, i is 0 to 2.0, j is 0 to 0.5 and
wherein the relative amount ratio of (3a+3c+2d+2e+g+2h+2i)/(2x12+2b) is from 0.90 to 1.10.

33. The process of claim 32 wherein the relative amount ratio of c to a is from 2.4 to 4.8, the relative amount ratio of c to g is from 3.3 to 4.8 and the relative amount ratio of (3a+3c+2d+2e+g+2h+2i)/(2x12+2b) is from 0.91 to 1.09.

34. The process of claim 32 wherein the catalyst has an X-ray diffraction pattern of diffraction peaks at the diffraction angles of 2θ, measured by using Cu Kα radiation, at 25.5, 26.6 and 28.0.

35. The process of claim 13 wherein g is in the range from 0.4 to 1.5.

36. The process of claim 13 wherein the compounds are dissolved and precipitation occurs at a temperature in the range of from 40° C. to 100° C.

37. The process of claim 36 wherein the compounds are dissolved and precipitation occurs at a temperature in the range of from 60° C. to 95° C.

38. The process of claim 13 additionally comprising aging for 2 to 24 hours before the liquid is removed.

39. The process of claim 38 wherein the aging is for 8 to 18 hours.

40. The process of claim 39 wherein the aging is for 5 to 10 hours.

41. The process of claim 13 wherein the solid is calcined at a temperature of 200-600° C. for 1-12 hours.

42. The process of claim 41 wherein the solid is calcined in two stages, one at a temperature of 150-400° C. for 1-5 hours and another at a temperature of 460-600° C. for 4-8 hours.

43. The process of claim 42 wherein the two-stage calcination is first at a temperature of 290-310° C. for 2 hours and second at a temperature of 460-500° C. for 6 hours.

44. The process of claim 13 wherein the solid is calcined in one stage at a temperature of 485° C. for 2 hours.

45. The process of claim 13 wherein prior to being calcined the solid is pretreated at a temperature of 300° C. for two hours.

46. A process of producing an unsaturated aldehyde from an olefin by catalytic oxidation comprising contacting the olefin and a molecular oxygen-containing gas in the presence of a catalyst of the formula:
Mo_{z}BiₐFe_{c}Cs_{g}Oₓ
wherein a is in the range from 0.1 to 1.5, c is in the range from 0.2 to 5.0, g is in the range from 0.1 to 1.5, x is determined by the valences of the other components and wherein the relative amount ratio of c to g is from 3.3 to 5.0, the relative amount ratio of c to a is from 2.0 to 6.0 and the relative amount ratio of (3a+3c+g)/(2×z) is from 0.90 to 1.10.

47. The process of claim 46 wherein the catalyst is of the formula:
Mo₁₂BiₐW_{b}Fe_{c}Cs_{g}MₘOₓ
wherein M is selected from cobalt, nickel, magnesium, zinc, potassium, rubidium, thallium, manganese, barium, chromium, cerium, tin, lead, cadmium and copper and
wherein m is 0 to 9 and b is 0 to 9 and
wherein the relative amount ratio of (3a+3c+g+Σvₙmₙ)/(2x12+2b) is from 0.90 to 1.10 with v being valence of each M and n being an integer for each M.

48. The process of claim 47 wherein the catalyst is of the formula:
MO₁₂BiₐW_{b}Fe_{c}Cs_{g}MₘM′_{m′}Oₓ
wherein M′ is one or more selected from antimony, phosphorus, boron, sulfur, silicon, aluminum, titanium, tellurium, vanadium, zirconium and niobium, m′ is in the range from 0 to 9.

49. The process of claim 48 wherein catalyst is of the formula:
Mo₁₂BiₐW_{b}Fe_{c}Co_{d}NiₑSb_{f}Cs_{g}MgₕZnᵢPⱼOₓ
wherein b is 0 to 4, d is 0 to 9, e is 0 to 9, f is 0 to 2.0, h is 0 to 1.5, i is 0 to 2.0, j is 0 to 0.5 and
wherein the relative amount ratio of (3a+3c+2d+2e+g+2h+2i)/(2x12+2b) is from 0.90 to 1.10.

50. The process of claim 49 wherein the relative amount ratio of c to a is from 2.4 to 4.8, the relative amount ratio of c to g is from 3.3 to 4.8 and the relative amount ratio of (3a+3c+2d+2e+g+2h+2i)/(2x12+2b) is from 0.91 to 1.09.

51. The process of claim 46 wherein the olefin is propylene and the aldehyde is acrolein.

52. The process of claim 46 wherein the olefin is isobutylene and the aldehyde is methacrolein and g is in the range from 0.4 to 1.5.

53. The process of claim 46 additionally comprising an inert gas.

54. The process of claim 53 wherein the inert gas is nitrogen.

55. The process of claim 46 wherein the oxygen is in a diluent gas.

56. The process of claim 55 wherein the diluent gas is nitrogen, a hydrocarbon which is gaseous under the process conditions or carbon dioxide.

57. The process of claim 46 wherein the reaction temperature is from 250 to 450° C.

58. The process of claim 46 wherein the reaction temperature is from 370 to 410° C.

59. The process of claim 46 wherein the reaction pressure is from 0 to 100 psig.

60. The process of claim 46 wherein the space velocity is from 800 to 8000 hr⁻¹.
